(19) United States
(12) Patent Application Publication
(10) Pub. No.: US 2014/0343340 A1
(43) Pub. Date: Nov. 20, 2014

(54) DEHYDROXYLATION OF POLYETHER POLYOLS AND THEIR DERIVATIVES USING A HALOGEN-BASED CATALYST

(71) Applicants: Raj Deshpande, Pune (IN); Paul Davis, Pune (IN); Vandana Pandey, Pune (IN); Nitin Kore, Solapur (IN); John R. Briggs, Midland, MI (US)

(72) Inventors: Raj Deshpande, Pune (IN); Paul Davis, Pune (IN); Vandana Pandey, Pune (IN); Nitin Kore, Solapur (IN); John R. Briggs, Midland, MI (US)

(21) Appl. No.: 14/364,721

(22) PCT Filed: Dec. 5, 2012
(86) PCT No.: PCT/US12/67837

§ 371 Date: Jun. 12, 2014

Related U.S. Application Data
(60) Provisional application No. 61/570,968, filed on Dec. 15, 2011.

(51) Int. Cl. C07C 1/22

(52) U.S. Cl. CPC C07C 1/22; C07C 2527/06
USPC 585/639

(57) ABSTRACT

Polyether polyols, derivatives and combinations thereof are converted to olefins under reductive or non-reductive dehydroxylation conditions, in the presence of a halogen-based catalyst. Derivatives include polyether polyols incorporated in polyurethanes. The process includes gas pressure from 1 psig (˜6.89 KPa) to 2000 psig (˜13.79 MPa), a temperature from 50° C. to 250° C., a liquid reaction medium, and a molar ratio of the starting material to halogen atoms from 1:10 to 100:1.

This application is a non-provisional application claiming priority from the U.S. Provisional Patent Application No. 61/570,968, filed on Dec. 15, 2011, entitled “DEHYDROXYLATION OF POLYETHER POLYOLS AND THEIR DERIVATIVES USING A HALOGEN-BASED CATALYST,” the teachings of which are incorporated by reference herein as if reproduced in full hereinbelow.

This invention relates generally to the field of dehydroxylation of polyether polyols. More particularly, it is a process to accomplish dehydroxylation of polyether polyols and mixtures and derivatives thereof to form olefins.

Polyols are compounds with multiple hydroxyl functional groups available for organic reactions. The main use of polymeric polyols is as reactants to make other polymers. For example, polyols can be reacted with isocyanates to make polyurethanes, a use which consumes most polyether polyols. These materials may be ultimately used to produce elastomeric shoe soles, fibers such as Spandex™, foam insulation for appliances such as refrigerators and freezers, adhesives, mattresses, vehicle upholstery, and the like.

Monomeric polyols, such as pentaerythritol, ethylene glycol and glycerin, often serve as the starting point for polymeric polyols. Naturally occurring polyols such as castor oil and sucrose may also be used to make synthetic polymeric polyols. These materials are often referred to as the “initiators” for the polymeric polyols. This means that they have at least one functional group that can be used as the starting point for a polymeric polyol. This functional group may be, for example, a hydroxyl or an amine. A primary amino group (—NH₂) often functions as the starting point for two polymeric chains, especially in the case of polyether polyols.

Polyether polyols, which account for a large majority of industrial polyol production, are frequently made by reacting epoxides, such as ethylene oxide or propylene oxide, with a multifunctional initiator in the presence of a catalyst. The catalyst is often a strong base, such as potassium hydroxide, or a double metal cyanide catalyst, such as zinc hexacyanocobaltate-t-butanol complex. Common polyether diols include polyethylene glycol; polypropylene glycol; and poly(tetramethylene ether) glycol.

Because polyols include highly-reactive hydroxyl groups by definition, they are among the candidates included for possible conversion to olefins. Researchers have addressed conversions of such hydroxyl containing materials, and mixtures thereof, in many ways. For example, United States Patent Publication (US) 2010/0077655 discloses the conversion of water soluble oxygenated compounds derived from biomass into C4+ liquid fuel hydrocarbon compositions via numerous steps incorporating, for example, dehydration, hydrogenolysis, and condensation. The multi-step process includes deoxygenation to form an oxygenate having the formula C₁₊O₁₋₃₊. These oxygenates comprise alcohols, ketones or aldehydes that can undergo further condensation reactions to form larger carbon number compounds or cyclic compounds. The catalysts proposed for the deoxygenation reaction are heterogeneous catalysts which consist of numerous metals and their combinations on a solid support. The support can be an acid, oxide, heteropolyacid, clay, or the like.

US 2010/0076233 discusses the conversion of oxygenated hydrocarbons to paraffins useful as liquid fuels. The process involves the conversion of water soluble oxygenated hydrocarbons to oxygenates, such as alcohols, furans, ketones, aldehydes, carboxylic acids, diols, triols, and/or other polyols, followed by conversion of the oxygenates to olefins via dehydration. Subsequently the olefins are reacted with C4+ iso paraffins to convert to C6+ paraffins. The reactions are conducted in the presence of a metal deoxygenation catalyst consisting of a support with any of various metals deposited thereon, either singly or in combinations. The support is selected from carbon, metal oxides, heteropolyacids, clays and their mixtures. The oxygenated hydrocarbons may originate from any source, but are preferably derived from biomass.

US 2010/0069691 discloses a method for the production of one or more olefins from the residue of at least one renewable natural raw material. The patent discusses the formation of ethylene and propylene via dehydration of ethanol and propanol. The ethanol and propanol are, in turn, prepared from biomass via fermentation of sugar (ethanol) and from syngas derived via gasification of biomass.

US 2009/0299109 discusses renewable compositions derived from fermentation of biomass. Fermentation produces C2-C6 alcohols, which can be dehydrated to olefins. The C2-C6 alcohols can be derived from biomass via fermentation or prepared via chemical routes involving catalytic hydrogenation. The dehydration of the alcohols is conducted in the presence of heterogeneous or homogeneous acidic catalysts.

US 2008/0216391 discloses the conversion of oxygenate hydrocarbons to hydrocarbons, ketones and alcohols useful as liquid fuels, such as gasoline, jet fuel or diesel fuel, and industrial chemicals. The process involves the conversion of mono-oxygenated hydrocarbons, such as alcohols, ketones, aldehydes, furans, carboxylic acids, diols, triols, and/or other polyols to C4+ hydrocarbons, alcohols and/or ketones, by condensation. The oxygenated hydrocarbons may originate from any source, but are preferably derived from biomass. The deoxygenation is conducted in the presence of a supported metal deoxygenation catalyst, while the subsequent condensation is conducted in the presence of an acid catalyst, preferably heterogeneous, such as an inorganic acid.

WO 2008/103480 discusses the conversion of sugars and/or other biomass to produce hydrocarbons, hydrogen, and/or other related compounds. The process involves the formation of alcohols or carboxylic acids from biomass. These are converted to hydrocarbons via decarboxylation or dehydration in the presence of hydrogen and either a metal or metal ion catalyst, or a basic catalyst.

Tetrahedron, Vol. 45, No. 11, pp 3569-3574, 1989 discloses vicinal diols and compounds containing vicinal diols being converted to olefins in the presence of aluminum triiodide in stoichiometric quantities.

Tetrahedron Letters, Vol. 23, No. 13, pp 1365-1366, 1982 discloses cis and trans vicinal diols being converted into olefins in a one-step reaction with chlorotrimethylsilane and sodium iodide. The mole ratio of sodium iodide is greater than the stoichiometric requirement, which indicates that the reagents are stoichiometric in nature.

Inorganic Chemistry, Vol. 48, pp 9998-10000, 2009 discloses methyltrioxorhenium (MTO) catalyzing the conversion of epoxides and vicinal diols to olefins with dihydrogen (H₂) as the reductant.

J. Am. Chem. Soc., Vol. 77, pp 365, 1955 discloses vicinal dihalides converted to olefins by reaction with iodide ion. The reaction is stoichiometric in the iodide and the starting materials are dihalides.

Chem. Commun., pp 3357, 2009 discloses the conversion of diols and polyols to olefins in the presence of formic acid.

In one aspect, this invention is a process for preparing an olefin comprising subjecting a starting material containing at least one polyether polyol, at least one derivative of a polyether polyol, or a combination thereof, to dehydroxylation conditions in the presence of a halogen-based catalyst containing at least one halogen atom per molecule thereof, which conditions include a reductive or a non-reductive gas, at an applied pressure of from 1 pound per square inch gauge (˜6.89 kilopascals) to 2000 pounds per square inch gauge (˜13.79 megapascals) or at autogenous pressure, a temperature within a range of from 50° C. to 250° C., a liquid reaction medium, and a ratio of moles of the starting material to moles of the halogen atoms ranging from 1:10 to 100:1; such that at least one olefin is formed.

A particular feature of the present invention is use of a halogen-based catalyst. As defined herein, a halogen-based catalyst contains at least one halogen atom and ionizes at least partially in an aqueous solution by losing one proton. It is important to note that the definition of halogen-based is applied to the catalyst at the point at which it catalyzes the dehydroxylation of the crude alcohol stream. Thus, it may be formed in situ in the liquid reaction medium beginning with, for example, a molecular halogen, e.g., molecular iodine (I₂), or may be introduced into the reaction as a halide acid, for example, as pre-prepared HI. Non-limiting examples include molecular iodine (I₂), hydroiodic acid (HI), iodic acid (HIO₃), lithium iodide (LiI), and combinations thereof. The term “catalyst” is used in the conventionally understood sense, to clarify that the halogen-based compound takes part in the reaction but is regenerated thereafter and does not become part of the final product. The halogen-based catalyst is at least partially soluble in the liquid reaction medium.

For example, in one non-limiting embodiment where HI is selected as the halogen-based catalyst, it may be prepared as it is frequently prepared industrially, i.e., via the reaction of I₂ with hydrazine, which also yields nitrogen gas, as shown in the following equation.

2 I₂+N₂H₄→4 HI+N₂ [Equation 1]

When performed in water, the HI must be distilled. Alternatively, HI may be distilled from a solution of NaI or another alkali iodide in concentrated hypophosphorous acid. Another way to prepare HI is by bubbling hydrogen sulfide steam through an aqueous solution of iodine, forming hydroiodic acid (which must then be distilled) and elemental sulfur (which is typically filtered).

H₂S+I₂→2 HI+S [Equation 2]

Additionally, HI can be prepared by simply combining H₂ and I₂. This method is usually employed to generate high purity samples.

H₂+I₂→2 HI [Equation 3]

Those skilled in the art will be able to easily identify process parameters and additional methods for preparing HI and/or other reagents falling within the scope of the invention. It is noted that sulfuric acid will not generally work for preparing HI as it will tend to oxidize the iodide to form elemental iodine.

As used herein the term “polyether polyol” is used to define a long chain molecule having multiple ether linkages, with hydroxyl end groups. Generally the molecular weight may range from 150 to 100,000 daltons (Da), and in particular embodiments may range from 1,000 to 50,000 Da. In still more preferred embodiments it may range from 5,000 to 20,000 Da. Polyether polyols may include, in non-limiting example, polyethylene glycol, polypropylene glycol, diethylene glycol, tetraethylene glycol dimethyl ether, tetraethylene glycol monomethyl ether, poly(tetramethylene ether) glycol, polyester-polyether polyols, and combinations thereof. Derivatives thereof may include, in non-limiting example, polyurethane compounds including, for example, polyurethane materials made from polyether polyols, wherein the reaction of an isocyanate group with a hydroxyl group has resulted in formation of a urethane linkage. Such may include true polyurethanes, as well as polyureas and polyurethane-ureas, which may be in the form, variously, of elastomeric materials, such as molded and slab foams, or rigid materials, such as both molded and spray foams. Combinations of any of the above are also comprehended. Collectively, these materials are referred to herein as the “starting material.”

In practicing the present invention the starting material and the catalyst are desirably proportioned for optimized conversion of the starting material to at least one olefin. Those skilled in the art will be aware without further instruction as to how to determine such proportions, but generally a ratio of moles of material to moles of halogen atoms ranging from 1:10 to 100:1 is preferred. More preferred is a molar ratio ranging from 1:1 to 100:1; still more preferably from 4:1 to 27:1; and most preferably from 4:1 to 8:1. Alteration of the proportion of the catalyst to starting material will alter the selectivity and conversion of products, but in general a starting material that is primarily propylene glycol will be converted predominantly to its corresponding olefin, propylene, while a starting material that is primarily ethylene glycol will be converted to its corresponding olefin, ethylene.

Temperature parameters employed in the invention may vary within a range of from 50° C. to 250° C., but are preferably from 100° C. to 210° C. Those skilled in the art will be aware that certain temperatures may be preferably combined with certain molar ratios of material and catalyst to obtain optimized olefin yield. For example, a temperature of at least 180° C. combined with a molar ratio of starting material to halogen atoms of 6:1 may result, in some embodiments, in particularly desirable yields. Other combinations of temperature and ratio of moles of starting material to moles of halogen atoms may also yield desirable results in terms of conversion of material and selectivity to desired alkenes. For example, with an excess of HI, temperature may be varied especially within the preferred range of 100° C. to 210° C., to obtain a range of conversion at a fixed time, e.g., 3 hours. Those skilled in the art will be aware that alteration of any parameter or combination of parameters may affect yields and selectivities achieved, and that routine experimentation to identify optimized parameters will be, as is typical, necessary prior to advancing to commercial production.

In certain particular embodiments the conditions may also include a reaction time, typically within a range of from 1 hour to 10 hours. While a time longer than 10 hours may be selected, such may tend to favor formation of byproducts such as those resulting from a reaction of the produced olefin, e.g., propylene or ethylene, with one or more of the starting material constituents. Byproduct formation may be more prevalent in a batch reactor than in a continuous process. Conversely, a time shorter than 1 hour may reduce olefin yield.

The inventive process may be carried out as either a reductive dehydroxylation or a non-reductive dehydroxylation. In the case of a reductive dehydroxylation, gaseous hydrogen may be employed in essentially pure form as the reductant, but also may be included in mixtures further comprising, for example, carbon dioxide, carbon monoxide, nitrogen, methane, and any combination of hydrogen with one or more the above. The hydrogen itself may therefore be present in the atmosphere, generally a gas stream, in an amount ranging from 1 weight percent (wt %) to 100 wt %.

Where a non-reductive dehydroxylation is desired, the atmosphere/gas stream is desirably substantially or, preferably, completely hydrogen-free. In this case other gases, including but not limited to nitrogen, carbon dioxide, carbon monoxide, methane, and combinations thereof, may be employed. Any constituent therefor may be present in amounts ranging from 1 wt % to 100 wt %, but the total atmosphere is desirably at least 98 wt %, preferably 99 wt %, and more preferably 100 wt %, hydrogen-free.

The hydrogen-containing (reductive) or non-reductive atmosphere is useful in the present invention at a gas pressure sufficient to promote conversion of, for example, molecular halogen to halide, for example, I₂ to an iodide, preferably hydroiodic acid (HI, also known as “hydrogen iodide”). The applied pressure is desirably from 1 psig (˜6.89 KPa) to 2000 psig (˜13.79 MPa), and preferably from 50 psig (˜344.5 KPa) to 200 psig (˜1.38 MPa). A gas pressure within the above ranges, especially the preferred range, is often favorable for efficient conversion of molecular halide to corresponding acid iodide. In many embodiments gas pressures in excess of 2000 psig (˜13.79 MPa) provide little or no discernible benefit and may simply increase cost of the process.

The conversion may be accomplished using many of the equipment and overall processing parameter selections that are generally known to those skilled in the art. Depending in part upon other processing parameters selected as discussed hereinabove, it may be desirable or necessary to include a liquid reaction medium. The starting material may function as both the compound(s) to be converted and the liquid reaction medium wherein the conversion will take place, or if desired, an additional solvent such as water, acetic acid, or another organic may be included. Acetic acid may help to dissolve the halogen formed as part of the catalytic cycle and act as a leaving group, thereby facilitating the cycle, but because esterification of the polyether polyol occurs, water is liberated. Conversely, while water may be effectively selected, particularly in the case of the non-reductive hydroxylation embodiment, selectivity may be thereby sacrificed. Organic solvents may be helpful in removing the accumulated water during the course of the reaction. In one embodiment, a carboxylic acid that contains from 2 carbon atoms to 20 carbon atoms, preferably from 8 carbon atoms to 16 carbon atoms, may be selected as a liquid reaction medium. Dialkyl ethers may also be selected.

### EXAMPLES

### General Experimental Procedure

Use a 300 milliliter (mL), High Pressure HASTELLOY™ C-276 Parr reactor with a glass insert as a reaction vessel. Charge 90 mL of acetic acid (S.D. Fine-Chem Ltd.) into the reactor. Add a known amount of polyether polyol and/or derivative thereof to the acetic acid. Add 4 mL of a 55% (weight/weight) aqueous solution of hydrogen iodide (HI) (Merck) or 3.73 gram (g) I₂ to the reactor, then close the reactor and mount it on a reactor stand. Flush void space within the reactor two times with gaseous nitrogen (200 psig (˜1.38 MPa). Feed H₂ into the reactor up to a pressure of 500 psig (˜3.45 MPa) and heat reactor contents, with stirring at a rate of 1000 revolutions per minute (rpm) up to a temperature of 210° C. Add sufficient additional H₂ to the reactor to increase pressure within the reactor up to 1000 psig (˜6.89 MPa). After 45 minutes of reaction time, remove a sample of vapor phase within the reactor using a gas sampling vessel. Analyze the sample via gas chromatography (GC) (Agilent 7890 with two thermal conductivity detectors (TCDs) and one flame ionization detector (FID)). Use a PoraPlot™ Q (Varian™ CP7554) column to separate carbon dioxide (CO₂), olefins and alkanes. Use a CP Wax (Varian™ CP7558) column to separate oxygenates and a molecular sieve (Molsieve™) (Varian™ CP7539) column to separate hydrogen, nitrogen and lower hydrocarbons. The reaction is continued in this fashion for a desired period of time. Based upon the vapor phase composition, calculate the mole percent (mol %) of polyol present in the crude stream corresponding to the olefin formed. The liquid phase is analyzed on GC (Liquid sample GC analysis is carried out using an Agilent 7890 gas chromatogram fitted with a split-splitless capillary injector with a split injector liner, tapered, low pressure drop with glass wool and flame ionization detector. The injection volume used is 1 microliter and split ratio is 1:20. The GC method uses a combined DB1701 and HP5 GC columns Samples are injected using an Agilent 7683B auto injector.

Calculate mole percent (mol %) conversion of material to olefin from vapor phase composition data according to the following equation:

$$mole \% = [ \frac{\begin{matrix}\frac{vol \%}{100} \times \frac{total pressure}{14.7} \times \\ \frac{volume of gas}{22400}\end{matrix}}{moles of materials} ] \times 100 \qquad [ Equation 4 ]$$

### Example 1

Using the above General Experimental Procedure with 0.19 moles of diethylene glycol (DEG), 0.029 moles of HI, temperature (T) of 210° C. and a time of 315 minutes (min), effect a 100% conversion of the DEG with 97, 1 and 2% selectivity to ethylene, ethane and carbon dioxide, respectively.

### Example 2

Replicate Example 1, except substitute 0.19 moles of polyethylene glycol (PEG, Mw 200) for the DEG, HI (0.029 mol), AcOH (90 mL), T=225 min, and H₂ (400 psig). This Example 2 effects 72% conversion of the PEG with a product stream selectivity of 98, 1 and 1% for ethylene, ethane and CO₂, respectively.

### Example 3

Replicate Example 1, except substitute 0.18 moles of polypropylene glycol (PPG, Mw 400), HI (0.029), AcOH (90 mL), T (210° C.), Time (270 min), H₂ (400 psig). After 270 minutes, selectivities are 45, 45, and 10%, respectively, to propylene, propane and CO₂, and total conversion is 31%, based on gas analysis.

### Example 4

Replicate Example 1, except substitute 0.26 moles of tetraethylene glycol dimethyl ether (TEGDME) for the DEG, HI (0.029), AcOH (90 mL), T (210° C.), time (360 min), H₂ (300 psig). After 360 minutes, conversion of TEGDME is 3%, with selectivity to ethylene, ethane and CO₂ being 98, 1 and 1%, respectively.

### Example 5

Replicate Example 1, except substitute 0.66 moles of tetraethylene glycol monomethyl ether (TEGMME) for the DEG; HI (0.029), AcOH (90 mL), T (210° C.), time (360 min), H₂ (300 psig). After 360 minutes, conversion of TEGDME is 26%, with selectivity to ethylene, ethane and CO₂ being 82, 18 and 0%, respectively.

### Example 6

A polyurethane foam is prepared by reacting a 5,000 M_{w} polyether (polypropylene glycol end-capped with ethylene oxide, PO/EO ratio 2.65) polyol with an isocyanate (toluene-2,4-diisocyanate) in a polyol:isocyanate ratio of 100:45.

An amount (7.79 g) of this polyurethane foam is processed according to Example 1 by substituting the foam for the DEG, and using HI (0.029 mole), AcOH (90 mL), T (210° C.), H₂ (400 psig), Time (315 min) After 315 min, 0.1 moles of C2 and C3 species are observed in the gas phase. The ethane and ethylene selectivities are 95 and 5%, respectively, and the propane and propylene selectivities are 87 and 13%, respectively.

1. A process for preparing an olefin comprising subjecting a starting material containing at least one polyether polyol, at least one derivative of a polyether polyol, or a combination thereof, to dehydroxylation conditions in the presence of a halogen-based catalyst containing at least one halogen atom per molecule thereof, which conditions include a reductive or a non-reductive gas, at an applied pressure of from 1 pound per square inch gauge (˜6.89 kilopascals) to 2000 pounds per square inch gauge (˜13.79 megapascals) or at autogenous pressure, a temperature within a range of from 50° C. to 250° C., a liquid reaction medium, and a ratio of moles of the starting material to moles of the halogen atoms ranging from 1:10 to 100:1; such that at least one olefin is formed.

2. The process of claim 1 wherein the polyether polyol has a molecular weight ranging from 150 to 100,000 Daltons.

3. The process of claim 1 or 2 where the polyether polyol is selected from the group consisting of polyethylene glycol, polypropylene glycol, diethylene glycol, tetraethylene glycol dimethyl ether, tetraethylene glycol monomethyl ether, and combinations thereof.

4. The process of any of claims 1 to 3 wherein the derivative is selected from the group consisting of polyurethanes, polyureas, polyurethane-ureas, polyester polyols, and combinations thereof.

5. The process of any of claims 1 to 4, wherein the applied pressure is from 50 psig (˜344.5 KPa) to 500 psig (˜3.45 MPa).

6. The process of any of claims 1 to 5, wherein the temperature is within a range of from 100° C. to 210° C.

7. The process of any of claims 1 to 6, wherein the ratio of moles of starting material to moles of halogen atoms ranges from 4:1 to 27:1.

8. The process of any of claims 1 to 7, wherein the ratio of moles of starting material to moles of halogen atoms ranges from 4:1 to 8:1.

9. The process of any of claims 1 to 8, wherein the halogen-based catalyst is selected from molecular iodine (I₂), hydrogen iodide (HI), and hydroiodic acid (HIO₃).

10. The process of any of claims 1 to 9, wherein the halogen-based catalyst is hydroiodic acid (HIO₃).
